(19) United States
(12) Patent Application Publication
(10) Pub. No.: US 2006/0277999 A1
(43) Pub. Date: Dec. 14, 2006

(54) Audiometer and method of hearing screening

(75) Inventor: Gabriel Raviv, Glenview, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: BIO-LOGIC SYSTEMS CORP., Mundelein, IL (US)

(21) Appl. No.: 11/150,970

(22) Filed: Jun. 13, 2005

(51) Int. Cl. A61B 5/12; A61B 5/00

(52) U.S. Cl. 73/585; 600/559

(57) ABSTRACT

An audiometer includes a data acquisition/transducer module, a signal processing module and a user interface. A probe is coupled to the data acquisition module. The signal processing module forms an otoacoustic emission stimulus signal for transduction to a patient via the data acquisition/transducer module and the probe and to detect a response to the stimulus. The audiometer operable to correlate the response to a hearing disability corresponding to a hearing loss at one of a plurality of auditory thresholds.

### TECHNICAL FIELD

This patent relates to an audiometer for screening for moderate or greater hearing disabilities and a method of screening using otoacoustic emission (OAE) techniques to rule out moderate or greater hearing loss.

### BACKGROUND

Conventional audiometry is performed by having a subject respond to acoustic stimuli by pressing a button, saying “yes”, or repeating words that may be presented in the stimulus. These tests are subjective in nature. Audiometry allows an audiologist to determine the auditory threshold of the subject, which is defined as the lowest intensity at which a sound can be heard. The audiologist evaluates the auditory threshold of a subject by using a stimulus that most commonly consists of a pure tone. The stimulus is presented via insert earphones, headphones, free field speakers or bone conduction transducers. The results are presented as an audiogram which shows auditory thresholds for tones of different frequencies. The audiogram is helpful for diagnosing the type of hearing loss a subject may have. The audiogram can also be used to fit a hearing aid and adjust the level of amplification of the hearing aid for subjects who require hearing aids.

One form of objective audiometry is otoacoustic emission (OAE) audiometry, and one form of OAE is distortion product otoacoustic emission audiometry (DPOAE). Otoacoustic emissions are small sounds generated within the cochlea that can be recorded in the external ear canal. When a stimulus is presented to the ear, mechanical and electrical events together can greatly enhance vibrations of the basilar membrane which is a necessary part for hearing and also produces otoacoustic emissions. Further, active processes in the cochlea may occur without any stimulus giving rise to spontaneous otoacoustic emissions. Outer hair cell function is affected by almost anything that can go wrong with the cochlea, and otoacoustic emissions are an extremely sensitive measure of cochlear functioning. Distortion product otoacoustic emissions (DPOAEs) are very low level stimulated acoustic responses to two pure tones presented to the ear canal. DPOAE measurement provides an objective non-invasive measure of peripheral auditory function and is used for hearing assessment. DPOAE screening is becoming a standard clinical practice to predict potential sensorineural hearing loss especially in newborns.

As noted, the response to the stimulus signal, typically in a range from about 50-70 dB, is a low level signal on the order of less than 10 dB. An OAE testing apparatus detects the response signal and verifies that the signal is a valid signal, e.g., by evaluating a signal-to-noise ratio of the detected signal. With a valid signal, the OAE testing apparatus may correlate the response to a healthy cochlea or to an indication of a potential hearing disability. Failing the OAE test, i.e., where the response is indicative of a potential hearing loss, generally results in a referral for additional screening and diagnosis to determine the existence and severity of any actual hearing disability. For infants and young children, a relatively high level of false positive is accepted as it is recognized that early detection and correction of hearing disability in children is critical to normal speech and learning development. Thus, most existing OAE testing apparatus provide a failing test indication where the indicated potential hearing loss is about 25 dB.

OAE has not found significant use in adult screening. An adult with mild hearing loss, in the range of about 25 dB to about 40 dB, may not have any significant difficulty functioning in society. Moreover, such a hearing loss may be acute and attributable to factors such as illness or earwax buildup. Because of the relatively low auditory threshold for failure of the standard OAE test, the adult with mild hearing loss would fail the OAE screening test and would likely be referred for additional screening and/or diagnosis. As a result, practitioners are reluctant to conduct OAE screening on adults because of the very high probability that the patient will fail the test, including those with only mild or moderate hearing loss who do not require additional diagnosis and/or corrective measures.

### BRIEF DESCRIPTION OF THE DRAWINGS

FIG. 1 depicts a functional block diagram of an audiometer adaptable to operate in accordance with one or more of the described embodiments;

FIG. 2 is a detailed block diagram of the probe component and the data acquisition component of the audiometer depicted in FIG. 1; and

FIG. 3 is a functional block diagram illustrating a software architecture that may be used in the audiometer depicted in FIG. 1.

### DETAILED DESCRIPTION

This patent describes embodiments of apparatus, systems, algorithms and methods of otoacoustic emission (OAE) screening and/or testing that are adaptable to the test subject. That is, the screening parameters are adaptable in view of the presentation of the test subject.

OAE generally and distortion product OAE (DPOAE) particularly have been recognized for a number years. While the herein described embodiments refer to DPOAE, it should be understood that the invention is not limited to a particular OAE technique. In DPOAE, two pure tones with frequencies ƒ₁ and ƒ₂ are presented to the cochlea. For best results, ƒ₂ is usually chosen as 1.2 ƒ₁. Due to the non-linearity of the ear, a very low level of distortion product of frequency 2 ƒ₁-ƒ₂ is generated in normal ears. The level of such DPOAE signal may be correlated to and provide an indication of the functionality of the ear and the existence of a potential hearing disability.

Conventionally, fast Fourier transform (FFT) may be used as the main signal processing tool to estimate the level of DPOAE signals. Alternatively, linear adaptive signal processing techniques may be employed. Such techniques generally offer better performance in terms of measurement time, which may be interpreted as higher noise immunity of adaptive techniques compared to FFT. Still other techniques, such as those described in U.S. patent application Ser. No. 10/698,801, filed Oct. 31, 2003, entitled “System and Method of Extraction of Nonstationary Sinusoids,” the disclosure of which is hereby expressly incorporated herein by reference, may also be used. The specific method of signal extraction is not critical to implementation of an apparatus, system, algorithm or method in accordance with the disclosed invention. The extracted signal is evaluated to determine that the response is valid, e.g., has a sufficiently high signal-to-noise ratio. A valid signal may then be evaluated to provide an indication of the existence of a potential hearing disability.

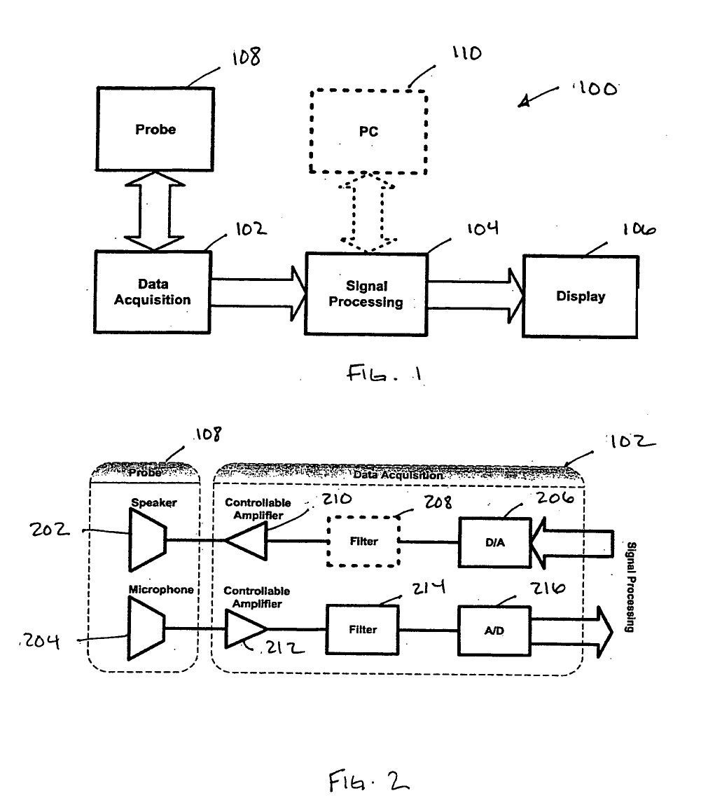

Referring to FIG. 1, an audiometer 100 includes three main modules: the data acquisition/transducers module 102, the signal processing module 104 and the user interface 106. A probe 108 is coupled to the data acquisition module 102, and a personal computer or other computational/data management device 110 may be coupled to the signal processing unit. The acquisition/transducer module 102 and the signal processing module 104 are described in more detail below. The user interface 106 may include a data entry device, such as a keypad, keyboard, touch screen, mouse or other pointer device, voice activation and the like for accepting commands, data input and actions from a user. The user interface 106 may also include a display and/or printer device for providing information to the user. The particular configuration of the user interface is not critical to operation of the audiometer 100, and the user interface function may also be provided by the personal computer 110. Thus, one of skill in the art given the nature of the data and commands to be accepted and the information to be provided to the user would be capable of specifying a suitable user interface. For example, the user interface may be a laptop or palm-type computing device with a graphic user interface and a keypad, mouse, joy-stick, touch pad and/or voice recognition input device. Furthermore, while shown as separate elements, the data acquisition module 102, the signal processing module 104, the user interface 106 and the probe 108 may be integrated into a single device, and potentially as a portable or handheld device.

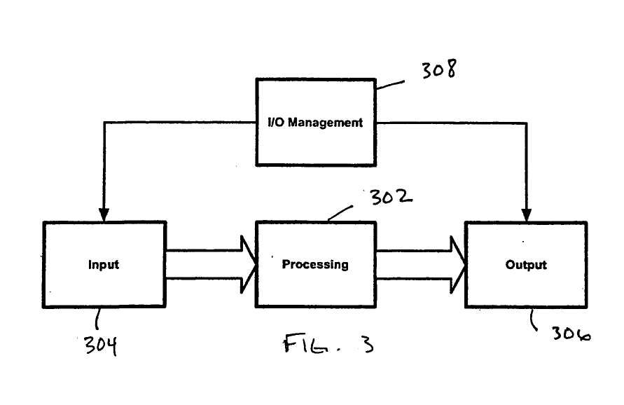

Data acquisition unit 102 is the medium between the processing unit 104 and the probe 108 which transmits and receives signals to the testing subject. For example, as shown in FIG. 2, the probe 108 may include a transducer 202 and a microphone 204 for transmitting and receiving signals in the human audible range. One of the main functions of the module 104 is to convert using a digital-to-analog converter 206 digital signals produced by the signal processing module 102 to stimulus signals, typically analog signals, which are then conditioned by optional filtering 208 and amplification 210 and converted to audio signals for transduction to the testing subject by the transducer 202. Conversely, the response signals detected by the probe, are conditioned by amplification 212 and filtering 214 and converted by the analog-to-digital converter 216 to digital signals to be processed by the signal processing module 102.

The signal processing module 102 of the audiometer 100 collects data via the analog-to-digital converter 216 representative of the sounds within the ear canal responsive to the stimulus. The detected data represents not only the response signal but other sounds including: the stimulus signal, artifacts and noise. The signal processing module 102 analyzes the data in order to extract and measure the response signal from the data.

A digital signal processor (DSP), or if the computational/architectural demand is low even a microcontroller, can be employed as the hardware platform of this unit. Signal processing is embedded as the software in such a hardware platform. Alternatively, and provided that the complexity of the signal processing algorithms remains low, signal processing unit may be implemented solely in hardware using programmable logic array (PLA) or field programmable gate array (FPGA) technology. In an ideal case, namely when the signal processing algorithm is not excessively complex, the hardware does not require a PC for its operation; however, interfacing to a PC is usually provisioned for data management.

FIG. 3 shows the main functions of the software embedded in the signal processing module 102. The software is essentially responsible for the generation of the stimulus signals and extraction of response signals, i.e., processing portion 302, as well as management of the input/output of data 304-308.

The processing portion 302 is adapted to provide passing/failing indications at a plurality of auditory thresholds. For example, the response to the stimulus may be indicative of a hearing loss of 25 dB auditory threshold or less as is typically used for infant and young children screening. A second passing/failing indication may be between about 40 dB-50 dB to provide an indication of a moderate or greater hearing loss. This later threshold will be more typically used for screening adults, but may also be used to screen other test subjects. Alternatively, the apparatus may be configured to provide a passing/failing indication selectable in the range from about 25 dB to about 50 dB. In this regard, the audiologist or other practitioner may select a screening auditory threshold based upon how the patient presents.

The user interface 106 may present a pre-screening configuration display that allows the audiologist/practitioner to select one of a number of predefined screening thresholds, e.g., 25 dB, 40 dB or 50 dB at which the audiometer 100 will provide a failing indication. The practitioner may further be presented the ability to select any auditory threshold less than about 50 dB at which the audiometer will provide a failing indication. After selection of the auditory screening threshold, the audiometer functions as described to conduct the OAE screening test to return the passing/failing result also via the user interface.

An audiometer 100 as described herein will prove to be a useful tool to the general practitioner, ear/noise/throat (ENT) specialist and/or skilled audiologist. For example, where an adult patient presents with a hearing loss complaint, the ENT specialist may determine the hearing loss is the result of earwax build up. After affecting a cleaning, it would be necessary to release the patient and have them report later whether they continue to experience hearing loss. With the audiometer 100, the ENT may affect a screening after the cleaning to determine if the hearing loss was properly attributable to the earwax build up or if some other condition is causing the loss. If the patient continues to screen with more than a mild hearing loss, the ENT may then confidently refer the patient to an audiologist or other specialist.

The audiometer 100 provides substantial flexibility. It is unnecessary to have more than one piece of screening equipment to screen for hearing disabilities at multiple auditory thresholds. The audiometer 100 is easily configured to screen for low or mild hearing loss at predetermined thresholds and/or to screen for virtually any auditory threshold below an indication of moderate hearing loss. The audiometer 100 may be a portable stand alone device, may be a stand alone device that couples with a computer or other data processing device for data management or may be an add-on peripheral to an existing audiometer tool. The functionality herein describe may further be implemented as a software program on any suitable media, such as electronic, magnetic or optical media, that may be installed in existing OAE audiometer devices and/or within a computer controlling the operation of an audiometer peripheral device.

The invention has been described in terms of several preferred embodiments and examples. One of skill in the art will appreciate that the invention may be otherwise embodied without departing from its fair scope, which is set forth in the subjoined claims.

1. An audiometer comprising:
operatively coupled: a data acquisition/transducer module, a signal processing module and a user interface;
a probe coupled to the data acquisition module; and
the signal processing module operable to form an otoacoustic emission stimulus signal and to receive a response signal, responsive to the stimulus signal via the data acquisition/transducer module and the probe; and to correlate the response signal to a hearing disability at one of a plurality of auditory thresholds.

2. The audiometer of claim 1, wherein the audiometer is operable to rule out existence of a hearing disability corresponding to a moderate or greater hearing loss.

3. The audiometer of claim 1, wherein the plurality of auditory thresholds comprise 25 decibels, 40 decibels and 50 decibels.

4. The audiometer of claim 1, wherein a selected one of the plurality of auditory thresholds comprises a threshold equal to or less than 50 decibels.

5. The audiometer of claim 1, wherein a selected one of the plurality of auditory thresholds comprises a threshold selected from the inclusive range of auditory thresholds from 25 decibels to 50 decibels.

6. A method of screening for a hearing disability comprising:
creating an otoacoustic stimulus signal;
transducing the signal to create an acoustic stimulus to create a transduced signal;
communicating the transduced signal to a test subject;
detecting a response to the transduced signal to obtain a measured response; and
determining a hearing disability corresponding to one of a plurality of auditory thresholds based upon the measured response.

7. The method of claim 6, comprising excluding the existence of a hearing disability corresponding to a moderate or greater hearing loss.

8. The method of claim 6, wherein the plurality of auditory thresholds comprise 25 decibels, 40 decibels and 50 decibels.

9. The method of claim 6, wherein a selected one of the plurality of auditory thresholds comprises a threshold equal to or less than 50 decibels.

10. The method of claim 6, wherein a selected one of the plurality of auditory thresholds comprises a threshold selected from the inclusive range of auditory thresholds from 25 decibels to 50 decibels.
